(19) United States
(12) Patent Application Publication
(10) Pub. No.: US 2012/0094351 A1
(43) Pub. Date: Apr. 19, 2012

(54) METHANIZATION METHOD FROM A LIQUID PHASE WHICH IS A COPRODUCT RESULTING FROM THE EXTRACTION OF A MAIN PRODUCT OBTAINED FROM A VEGETABLE RAW MATERIAL

(75) Inventors: John Mahler, GB-London (GB); Claude Delporte, Rueil Malmaison (FR); Sarah Maillard, Saint Germain En Laye (FR)

(73) Assignee: ONDEO INDUSTRIAL SOULUTIONS, VAUCRESSO (FR)

(21) Appl. No.: 13/266,362

(22) PCT Filed: Apr. 27, 2010
(86) PCT No.: PCT/IB10/51829

§ 371 Date: Nov. 30, 2011

(30) Foreign Application Priority Data
Apr. 30, 2009 (FR) 0902116

(51) Int. Cl. C12P 5/02

(52) U.S. Cl. 435/167

(57) ABSTRACT

The invention relates to a methanization method from a liquid phase which is a coproduct resulting from the extraction of a main product obtained from a vegetable raw material, wherein the weight content of the suspended material (MES) in said liquid phase is lower than 0.25%, and the methanization processing of said liquid phase is carried out using a very high yield digester.

### TECHNICAL FIELD OF THE INVENTION

The present invention relates to a process of methanization from a liquid phase which is a coproduct resulting from the extraction of a main product obtained from a vegetable raw material.

### TECHNICAL BACKGROUND

The invention relates, for example, to a process of methanization from a product extracted from a fermented wort, in particular from the vinasse resulting from the distillation of a fermented wort of a vegetable raw material in the context of alcohol production, from a vegetable raw material, in particular from starchy plants and/or from sacchariferous plants and/or from lignocellulosic plants.

Such an alcohol production process is, for example, of the type that comprises the following steps:
preparation, with the vegetable raw material, of a pulp suitable for fermentation, called a wort,
fermentation of the wort thus prepared,
separation by filtration, in particular by filtration-pressing, of the solid phase and of the liquid phase of the fermented mixture,
distillation of said liquid phase of the fermented wort, for example in a distillation column, with recovery of the alcohol at the top of the column and of the vinasse at the bottom of the column.

An example of such a process is known from patent application WO-A1-2008/003692 in the name of Mr John MAHLER, which targets in particular the production of alcohol from starchy vegetable raw material.

The process can also provide for the recovery of the phlegmas obtained in the rectification step.

The liquid phase of the vinasse is a coproduct of alcohol production, the potential of which can be exploited, for example with a view to producing thermal and/or electrical energy, by subjecting it to methanization processing.

In such an alcohol production process, the technique used to separate the coproduct in the liquid phase is such that the content by weight of suspended solids in the liquid phase of the vinasse before methanization is less than 1%, particularly 0.25% and more particularly 0.2%, or else preferably about 0.1% (analysis according to standard NF EN 872).

Generally, this may involve the methanization of the vinasse obtained in particular in the context of the production of methyl alcohol, of ethyl alcohol, of butyl alcohol, and the like, from a starchy and/or sacchariferous and/or cellulosic vegetable raw material

(VRM). In addition to these various types of alcohol, the methanization according to the invention can also be applied to the coproduct in the liquid phase resulting from the production of other products, for instance the production of beer, of cider, of champagne, of vinegar, of soya-derived products, of amino acids, of citric acid, of succinic acid and other acids, the production of yeasts, of vitamins, of antibiotics, of sauerkraut or of cheeses, and from fermentation from vegetable raw material containing sugary ingredients, starchy ingredients and cellulosic ingredients.

The processing of vinasses with a high COD (chemical oxygen demand) and a. high nitrogen, phosphorus, suspended solids, calcium, magnesium, potassium, sulfate and sodium load, and the like, is difficult and complex. The volumes to be used result in large processing works and in high investment costs for the production equipment.

In addition, the high concentrations of calcium and magnesium promote precipitation phenomena in the processing equipment and, depending on the pH, can considerably slow down the activity of the biomass. Furthermore, nitrogenous, phosphorus-containing, potassium, sulfate and sodium compounds, and the like, can have inhibitory effects on the biological activity.

The methanization step is thus relatively long, and can last more than 30 days, which is extremely disadvantageous for large-scale industrial processing. In addition, the residual pollution of the effluents resulting from the methanization is considerable and the equipment must be completed with an aerobic biological processing stage that results in large reactor volumes and in significant production of biological sludge.

Document WO 2004/113549 (WILKENING) also relates to a process for producing ethanol and methane from biomass. The duration of the methanization step remains long. Although the WILKENING process makes reference to an SS content equal to 1%, it does not propose any industrial solution for achieving such a level. Furthermore, tests carried out by the applicants have shown that the low yields obtained for the methanization do not make it possible to envision a cost-effective industrial implementation, particularly from the viewpoint of the durations necessary for the methanization.

The objective of the invention is in particular to reduce the time required for the methanization step and also to decrease the production of sludge, by offering possibilities of recycling the effluents resulting from the methanization, in particular in the form of water that can in particular be used in the process for producing the main product, and thus to reduce the consumption of water used for producing the wort.

### BRIEF SUMMARY OF THE INVENTION

With this objective, the invention proposes a process of methanization, from a liquid phase which is a coproduct resulting from the extraction of a main product obtained from a vegetable raw material, characterized in that the content by weight of suspended solids (SS) in said liquid phase is less than 0.25%, and the methanization processing of said liquid phase is carried out by means of a very high yield digester.

For the definition of a very high yield digester, it is a digester of which the yield is greater than 90%.

Preferably, the content by weight of suspended solids (SS) in said liquid phase is less than 0.2%, in particular less than or equal to 0.1%.

The very high yield digester may comprise a bed of fluidized, recirculated or expanded granular sludge in one or more stages or of flocculated sludge, according to an upflow of the liquid phase.

The very high yield digester may also be:
a reactor containing a fluidized bed of biomass (cultures attached to a mobile free support);
or a reactor containing a fixed bed of biomass (culture attached to an immobile support).

The invention thus proposes a process of methanization, at very high yield, from a liquid phase which is a coproduct resulting from the extraction of a main product obtained from a vegetable raw material, the content by weight of suspended solids (SS) in said liquid phase being less than 0.25%, advantageously less than 0.2%, in particular less than or equal to 0.1%.

According to the process, the yield for elimination of the carbonaceous pollution (COD and BOD_{S}) is about 90% and up to approximately 97%.

The very high yield obtained during the methanization makes it possible in particular to remove virtually all the soluble biodegradable pollution, which makes it possible to directly envision processing using membrane devices, and in particular using the reverse osmosis technique.

Conversely, the implementation of reverse osmosis at the end of a “conventional” methanization does not enable processing by reverse osmosis, in particular owing to clogging of the membranes, or else this requires additional intermediate biological processing, between the exiting of the methanization and the entry into the reverse osmosis. Furthermore, the implementation of such intermediate finishing processing itself produces sludge for which it is then necessary to envision additional removal or recycling solutions.

According to other features of the process according to the invention:
the methanization processing is carried out according to a pulsed feed of said liquid phase in a digester of the “Anapulse” type;
the methanization processing is carried out by means of a reactor containing a fluidized bed of biomass in a digester of the “Anaflux” type;
the methanization processing is carried out by means of a reactor containing a fixed bed of biomass in a digester of “Anafiz” type;
at least one effluent resulting from the methanization is subjected to a stripping and decarbonation step, and at least one fraction of the decarbonated effluent obtained is recycled to the top of the methanization;
said recycled fraction is included between 0 to 400% by weight of the vinasses and of the phlegmas;
the fraction of the decarbonated effluent which is not recycled to the top of the methanization is subjected to a filtration suitable for stopping particles having sizes of about 0.2 μm, in particular bacteria;
said filtration of the fraction of the stripped and decarbonated effluent which is not recycled is carried out by reverse osmosis, in particular for recycling directly to the production process or various production units;
before said filtration, said fraction of the decarbonated effluent is subjected to biological processing of the carbonaceous and/or nitrogenous pollution, and/or to physicochemical processing of the phosphorus-containing pollution;
carbon dioxide (CO₂), in particular carbon dioxide produced during the process, is used:
for neutralization of the lime in order to produce carbonate of lime,
for the partial replacement of at least one acid used for said extraction of the main product;
40 to 95% of the effluent after methanization, stripping and decarbonation processing and filtration can be recycled in the form of bacteria-free water;
in addition to the constituent liquid filtrate of the recycling water, the filtration by reverse osmosis produces a retentate which subsequently undergoes an “evaporation-concentration” step, and the condensates recovered after evaporation-concentration can be used in the form of water.

### BRIEF DESCRIPTION OF THE FIGURES

Other features and advantages of the invention will emerge on reading the description which follows, given by way of nonlimiting examples, for the understanding of which reference will be made to the attached drawings in which:

FIG. 1 is a diagrammatic representation of the main large groups of operations generally included in a first example of a process for producing a main product and a coproduct in the liquid phase capable of being methanized, the separation of the liquid phase occurring before the extraction of the main product;

FIG. 2 is a diagrammatic representation of the main large groups of operations generally included in a second example of a process for producing a main product and a coproduct in the liquid phase capable of being methanized, the separation of the liquid phase occurring after the extraction of the main product;

FIG. 3, in the form of three consecutive and linked parts 3A, 3B and 3C, is a detailed representation of the main groups of operations included in an example of a process for producing a main product and a coproduct in the liquid phase capable of being methanized, the separation of the liquid phase occurring before the extraction of the main product, and of the main groups of operations included in an example of a process for methanization and processing of the effluents of the methanization in accordance with the teachings of the invention.

### DETAILED DESCRIPTION OF THE FIGURES

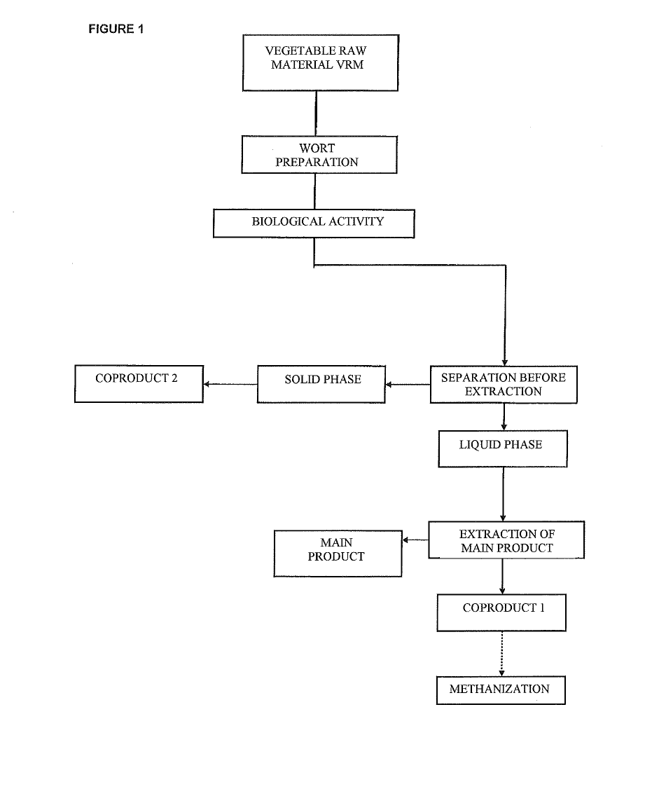

A process for obtaining a main product obtained upstream of the methanization has been represented in FIG. 1. Said main product is, for example, ethanol, bioethanol, etc.

This process comprises in particular, starting from VRM, a step of preparation of a wort, a biological activity step (for example a step of transformation and production of the main product) and a step of separation before extraction of the liquid and solid phases.

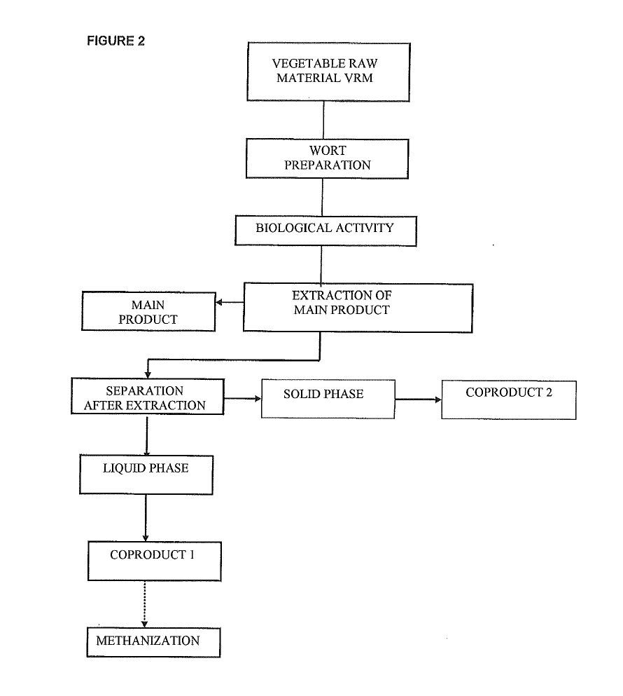

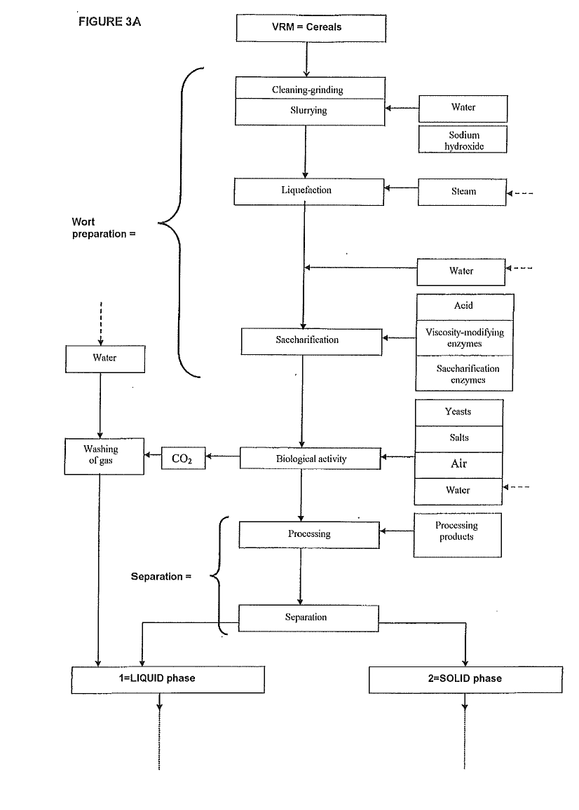

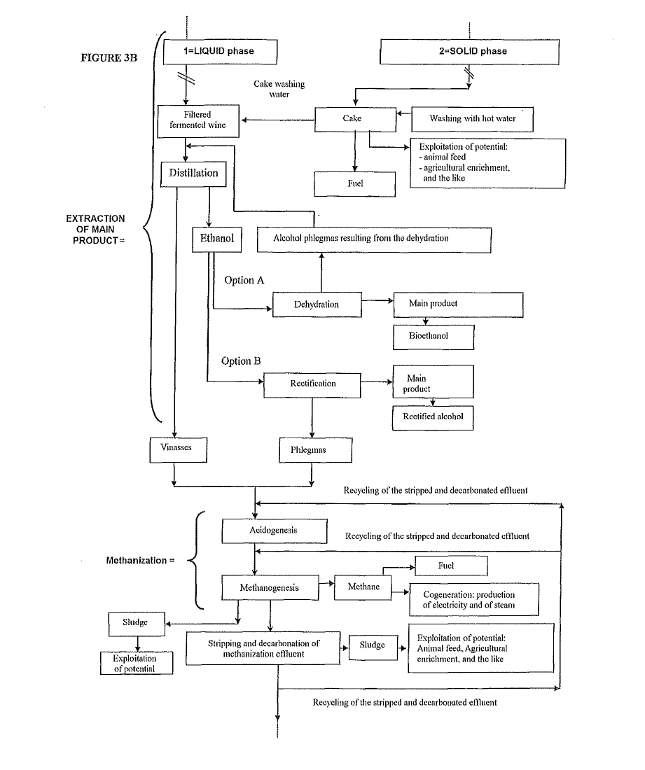

The “separation” step, whether it occurs before or after (see FIG. 2) the extraction of the main product, results in the production of two coproducts:

a main coproduct or coproduct 1, which is the liquid phase resulting from the separation step;

a secondary coproduct or coproduct 2, which is the solid phase resulting from the separation step.

Whether the separation takes place before or after the extraction of the main product, the separation technique, for example according to the teachings of the abovementioned John MAHLER document, the main feature of the coproduct 1 (liquid phase) is its content by weight of suspended solids (SS).

The solid phase (COPRODUCT 2)/liquid phase (COPRODUCT 1) separation is carried out under conditions such that the content by weight of suspended solids (SS) in the liquid phase (COPRODUCT 1) before methanization is less than 0.25%, and preferably less than 0.2%, in particular less than or equal to 0.1%.

FIGS. 1 and 2 are two diagrams illustrating the two versions (before and after extraction) of the separation with a view to the production of the main coproduct or coproduct 1 which will subsequently undergo the methanization process.

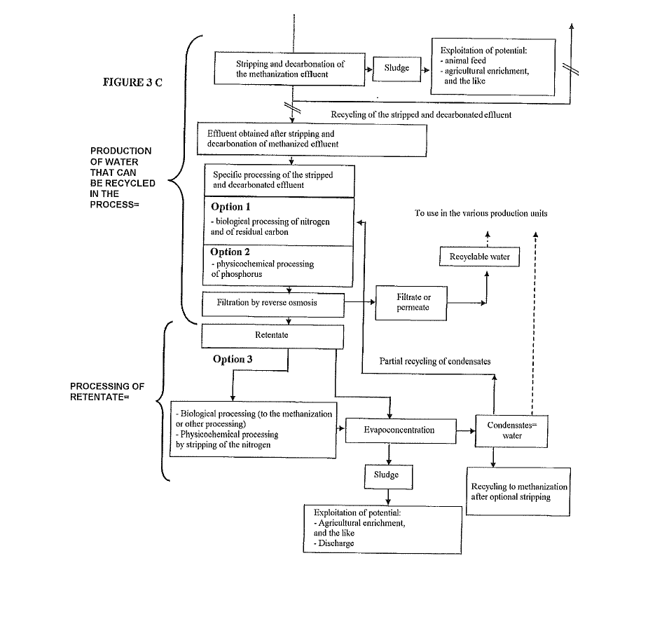

FIG. 3 is a detailed overall representation which reproduces “upstream” and in a more detailed manner an example of the production by extraction of the main product and the separation of the two coproducts in an example in which this separation takes place before the extraction, i.e. corresponding to the diagrammatic representation of FIG. 1.

FIG. 3 contains the vegetable raw material VRM which, in this case, consists of cereals.

The preparation of the wort indicated in FIG. 1 is detailed in FIG. 3 in the form of the steps of cleaning-grinding the cereals, of slurrying by adding water, liquefaction enzymes and sodium hydroxide, of liquefaction by means of steam, and of saccharification by adding acid, viscosity-modifying enzymes and saccharification enzymes.

The liquefaction and the saccharification are aimed at obtaining a fermentable sugar.

After this preparation of the wort, the biological activity step is carried out, which, by way of example, is a process of fermentation by adding yeasts, salts, air and water.

After this development, or biological activity, by fermentation, the separation steps are carried out (in this case before extraction).

The separation of the liquid and solid phases comprises a first “processing” step (Mahler process) by adding products for modifying the pH and filtration adjuvants, for example based on polymers, and then a step of actual physical separation, by means, for example, of a filter press, of the two liquid and solid phases, the separation being, for example, by filtration and pressing.

Indicated in the right-hand part of FIG. 3A are various products and components necessary for the various steps or substeps of wort preparation, of development and production of the main product (biological activity) and of separation.

In the left-hand part of FIG. 3A, it has been indicated that the fermentation produces carbon dioxide (CO₂) which can be washed (by means of water) so as, in a known way, to recover a part of the alcohol contained in the carbon dioxide resulting from the fermentation.

If this washing of the CO₂ resulting from the fermentation is carried out, the gas washing water, loaded with recovered alcohol, is added to the liquid phase resulting from the separation, which is the main coproduct or coproduct 1 resulting from the separation process.

Downstream of the separation, and moving on to part 3B of FIG. 3, by way of observation, in this particular example, the liquid phase is also called filtered fermented wine.

The solid phase, or coproduct 2, is also called cake. This coproduct 2 is, for example, of use as a fuel.

Before being used as a fuel, the coproduct 2 can undergo washing with hot washing water, this washing water for the coproduct 2 then being added to the coproduct 1 (Filtered Fermented Wine), this taking place before the extraction of the main product.

The various processing operations on the Filtered Fermented Wine (coproduct 1) to which the cake (coproduct 2) washing water has optionally been added will now be described.

The next main step is therefore the step of extraction of the main product(s) which in this case is, by way of example, a distillation step.

According to a first option A, the main product can undergo a dehydration, of which the source of energy is, for example, pressurized steam, in order to produce bioethanol. The alcoholic phlegmas resulting from the dehydration can be reintroduced upstream of the distillation.

According to another option B, the main product resulting from the distillation undergoes a rectification, for example by means of pressurized steam, in order to produce rectified alcohol.

In addition to the rectified alcohol, the rectification results in the production of nonalcoholic phlegmas which will subsequently be used and reintroduced upstream of the methanization.

The two options A and/or B of dehydration and of rectification can be combined.

In addition to the production of the final main product or final main products (in this case bioethanol and/or rectified alcohol), the distillation results in the production of liquid vinasses.

It is these vinasses, optionally augmented by the phlegmas resulting from the rectification, which will undergo the methanization.

The methanization process mentioned in FIGS. 1 and 2 essentially consist, in the known manner, of a first “acidogenesis” step and then of a second “methanogenesis” step.

The methanogenesis step produces biogas, a processed effluent and a small amount of sludge the various uses of which will be commented upon subsequently.

Of course, the methanization results, in the main, in the production of methane which can, for example, be used as a fuel for the purpose of producing energy.

In addition to the sludges and the methane, the methanization produces a processed effluent which, subsequently, is in this case subjected to a “stripping and decarbonation” step.

The stripping and decarbonation step results in the production of sludges and in a decarbonated effluent.

Other than the sludges, the effluent thus stripped and decarbonated is subsequently completely or partially subjected to a reverse osmosis filtration step.

Indeed, a first part or fraction of the stripped and decarbonated effluent is immediately recycled upstream of the methanization.

This recycled fraction is included between 0 and 400% by weight of the vinasses and of the phlegmas.

The fraction of the stripped and decarbonated effluent which is not recycled upstream or to the top of the methanization is subjected to a filtration. Preferably, the filtration is carried out by reverse osmosis on membranes.

In order to obtain water to be recycled that does not contain any bacteria that can cause an infection and blocking of the specific biological activity of the production of the main product, the stripped and decarbonated methanization effluent is subjected to a filtration which is suitable for stopping bacteria and particles having sizes of about 0.2 μm and above.

This filtration process, in particular by reverse osmosis, for stopping the bacteria, gives, as filtrate, water of very good quality from which the salts have been removed and which can be recycled to the main production process in particular without risk of infection that can block the biological activity.

As an option (options 1 and 2) and prior to the reverse osmosis filtration, the fraction of the stripped and decarbonated effluent is subjected to specific processing of the effluent, including biological processing of the carbon so as to remove the residual carbonaceous pollution (option 1) and physicochemical processing of the phosphorus (option 2).

The filtrate, or permeate, obtained by reverse osmosis filtration is a water that can be recycled and in particular can be used as water in the wort preparation, biological activity and separation steps. The evapoconcentration (evaporation/concentration) condensates described hereinafter may also be used at this level or in other production units.

This filtration is suitable for stopping particles having sizes of about 0.2 μm (0.2 micron), in particular, for example, bacteria.

In addition to the constituent liquid filtrate of the recycling water, the reverse osmosis also produces a concentrate or retentate which may subsequently, for example, undergo an “evaporation-concentration” or “evapoconcentration” step, in particular by steam heating.

Prior to the evapoconcentration (option 4), all or part of the concentrate resulting from the reverse osmosis can undergo, as an option (option 3), a step of biological processing of the carbonaceous and nitrogenous pollution (also called nitrification-denitrification) and/or a step of physicochemical processing of the phosphorus-containing pollution (dephosphatation).

All or part of the concentrate having undergone these biological and/or physicochemical processing steps is then subsequently subjected to the evapoconcentration.

The evapoconcentration results first in the production of (liquid) condensates which can be used again as recycling water in the various process steps. A part of the liquid condensates can also be recycled upstream of the reverse osmosis filtration step.

In addition to the liquid condensates, the evapoconcentration produces sludge. The potential of this sludge may be exploited (for example for the enrichment of agricultural soils) or it may be discharged.

In the example described, the liquid condensates resulting from the evapoconcentration are used directly in the form of water in the production units and/or are recycled upstream of the reverse osmosis filtration.

By way of a variant that is not represented, the liquid condensates resulting from the evapoconcentration can undergo a stripping step and, at the end of this stripping, a part of the condensates is used as recycled water in the production process, another part possibly being recycled upstream of the reverse osmosis filtration and/or another part possibly also being recycled upstream of the methanization.

Various steps mentioned above result in the production of sludge. This may, for example, be methanization sludge, decarbonation sludge and evapoconcentration sludge, etc.

This sludge can, for example, be dehydrated, according to various known techniques.

The sludge can thus be burnt, either “at a loss” by incineration, or by combustion with a view to recovering energy.

The potential of the sludge can also be exploited (production of components for the enrichment of agricultural soils, sludge methanization and biogas production, or the like) or the sludge can be discharged.

According to the invention, the methanization process which comprises the acidogenesis step and a methanogenesis step is carried out according to an upflow through a bed of sludge attached to a support or a bed of flocculated or granular sludge made up of compact aggregates of bacteria and/or of granules having an average diameter generally greater than 0.1 mm. The liquid phase (COPRODUCT 1) is introduced into a reactor, for example in the bottom part, via a network of perforated pipes.

When flocculated or granular sludge is used, the liquid-phase vinasse feed may advantageously be pulsed.

The methanization reactor is closed for an anaerobic reaction. A biogas outlet is provided in the upper part. The biogas composed mainly of methane is used, for example, for producing electrical energy, using a turbine or any other appropriate device, and/or for producing heat.

For example, a reactor, also called digester, of this type is known under the name “Anapulse”, and is described in particular in the Memento Technique de l'Eau [Technical memorandum on water] from Degremont, 10^{th} edition, pages 975-976. Such a reactor is normally envisioned for brewery, sweetened beverage, sugar refinery, starch factory, paper mill, yeast factory, etc., effluents.

The methanization can also be carried out in single-stage or two-stage, fluidized, recycled or expanded granular sludge reactors.

The methanization can also be carried out, for example, in a reactor or digester of the “Anaflux” type, which is a digester comprising cultures attached to a support that is free in the reactor. A reactor, also called digester, of this type is described in particular in the Memento Technique de l'Eau [Technical memorandum on water] from Degremont, 10^{th} edition, pages 977-978.

Such a reactor is usually envisioned for brewery, sweetened beverage, sugar refinery, starch factory, paper mill, yeast factory, etc., effluents.

The methanization can also be carried out, for example, in a reactor or digester of “Anafiz” type, which is a reactor or digester comprising a culture attached to a packing (for example made of plastic, polypropylene, polyethylene), which is ordered or loose.

A reactor, also called digester, of this type is described in particular in the Memento Technique de l'Eau [Technical memorandum on water] from Degremont, 9^{th} edition, pages 753-756.

By virtue of the thorough solid phase (COPRODUCT 2)/liquid phase (COPRODUCT 1) separation processing, the methanization can be carried out industrially, for example in a granular-sludge or fluidized-bed reactor, which makes it possible to considerably reduce the duration of the methanization. This duration becomes less than approximately six days and, for example, about three to four days, whereas it is generally greater than twenty days, and could reach thirty days and more, for the processing by methanization of the liquid phase or vinasse as it is on leaving the distillation column.

The effluent resulting from the methanization is subjected to the stripping and decarbonation step in a closed and deodorized tank. A stream of gas, generally air, is blown into the bottom of the tank in order to eliminate the carbon dioxide (CO₂) and to oxidize the hydrogen sulfide gas (H₂S) that may be present in the effluent by entraining them to deodorization processing. The effluent is subsequently decarbonated.

The stripping-decarbonation processing or step makes it possible in particular to recycle, to the digester, an effluent which has a low load in terms of calcium and magnesium, which would have a tendency to be deposited on the granular sludge or the biomass supports, on the biomass support materials and the sludge aggregates, with, as a result in this case, a decrease in the carbonaceous pollution elimination yield subsequent to biological and hydraulic problems.

The invention thus provides a solution to several technical problems encountered during methanization according to the prior art:
the COD and BOD₅ elimination yields obtained in the methanization reactors were insufficient, problems related to inhibition phenomena because of high concentrations, greater than or above the toxicity thresholds, of certain components such as SS, anions or cations, did not make it possible to reach favorable energy balances for the equipment;
the management of the associated liquid and solid waste was complex and expensive;
the size of the equipment and the investments were considerable owing to the long residence time in the methanization reactors;
precipitation phenomena occurred in the processing works.

All these problems and drawbacks did not enable the implementation, on the industrial scale, of a technically and economically viable vinasse-processing solution.

Furthermore, the biogas produced is “clean”, in particular from the viewpoint of hydrogen sulfide H₂S. The reduction in sulfates in the vinasses makes it possible to avoid the harmful aspects of H₂S, in particular during methanization (odor, corrosion, and pollution in the subsequent form of SO₂).

As regards H₂S, it is a part of the substrate which normally serves to produce methane, which is “used” for the conversion of SO₄²⁻ into H₂S.

A gain in yield is thus made during the methanization by devoting a greater part of the substrate for the production of methane.

The abbreviations used in the description are explained hereinafter:

Alkalinity: (NF EN ISO 9963-1)

VFAs: volatile fatty acids (chromatographic analysis)

Ca: calcium (NF EN ISO 11885)

BOD₅: biochemical oxygen demand in five days (NF EN 1899-1, NF EN 1899-2)

COD: chemical oxygen demand (NFT 90-101, ISO 6060: 1989)

K⁺: potassium (NF EN ISO 11885, NF EN ISO 14911)

SS: suspended solids, total suspended solids (NF EN 872, NFT 90-105-2)

Mg: magnesium (NF EN ISO 11885)

DM: dry matter (NF U 44-171)

VSS: volatile suspended solids (NF U 44-171)

Na⁺: sodium (NF EN ISO 11885, NFT 90-019, NF EN ISO 14911)

TN: total nitrogen (NF EN ISO 11905-1)

NH₄: ammonium (NF T 90-015-1, NF T 90-015-2)

NO₂: nitrite (NF EN ISO 13 395, NF EN 26777)

NO₃: nitrate (NF EN ISO 13 395, NF EN ISO 10304-1)

ortho-P: orthophosphate (NF EN 1189, NF EN ISO 6878)

TP: total phosphorus (NF EN 1189, NF EN ISO 6878)

pH: hydrogen potential (NF T 90-008)

SO₄²⁻: sulfate (standard NF EN ISO 10304-1)

TH: total hardness (NFT 90-003)

PA: P-alkalinity (NF EN ISO 9963-)

TA: Total alkalinity (NF EN ISO 9963-)

Table 1 describes, by way of example, the composition of the liquid phase (COPRODUCT 1) after separation upstream of the extraction and resulting from the production of bioethanol from starchy, sacchariferous or lignocellulosic plants.

TABLE 1

| Parameters | COPRODUCT 1 |
|---|---|
| pH | 2 to 10 |
| TH (° F.) | 0 to 1000 |
| PA (° F.) | 0 to 500 |
| TA (° F.) | 0 to 500 |
| Ca (g/l) | 0 to 4 |
| Mg (g/l) | 0 to 2.5 |
| COD (g/l) | 1.5 to 300 |
| SS (g/l) | 0.1 to 5 |
| NH₄⁺ (g/l) | 0 to 2.5 |
| TN (g/l) | 0 to 5 |
| TP (g/l) | 0 to 2.5 |
| ortho-P (g/l) | 0 to 2 |
| NO₂ (mg/l) | 0 to 500 |
| NO₃ (mg/l) | 0 to 500 |

Table 2 presents the operating conditions for carrying out the steps of processing by acidogenesis and by methanogenesis

TABLE 2

| | Processing by | Processing by |
|---|---|---|
| Parameters | acidogenesis | methanogenesis |
| pH | 4-7 | 6-9 |
| Mesophilic t (° C.) | 30-40° C. | 30-37° C. |
| Thermophilic t (° C.) | 38-60° C. | 50-60° C. |
| Conditions | anoxic/anaerobic | anaerobic |
| Residence time | 5 to 24 h | 1 to 6 days |
| Methane production | — | 0.25 to 0.35 Nm³/kg |
| | | COD_{eliminated} |
| VFAs (g/l) | 1 to 25 | <0.5 on leaving the |
| | | digester |

The results obtained according to the invention after processing by methanization, stripping and decarbonation are the following:
About 90% reduction in COD and in BOD₅ and up to approximately 97%.
Methane production between 0.25 and 0.35 Nm³/kg COD eliminated.

After methanization, the effluent is stripped in order to eliminate the CO₂ and to increase the pH and thus to reduce the amounts of reactant to be introduced for the decarbonation.

After stripping, lime is added (0.1 to 5 g/l) so as to increase the pH up to 10-11.

The mixture is stirred for 0.5 to 5 h. After reaction, a decanting step makes it possible to separate the flocs formed, which will settle to the bottom of the tank, and the decarbonated effluent is recovered at the surface.

The concentrations of the effluent resulting from the processing by methanization after recycling of the stripped and decarbonated effluent (0 to 400% by weight of vinasses and of phlegmas) are given in table 3 which follows.

TABLE 3

| | Effluent resulting from the |
|---|---|
| | methanization after recycling of the |
| Parameters | stripped and decarbonated effluent |
| pH | 6.5-8.5 |
| Temperature (° C.) | 30-37 |
| TH (° F.) | 50-200 |
| PA (° F.) | 0-150 |
| TA (° F.) | 50-500 |
| COD (mg/l | 500-7500 |
| SS (mg/l) | 50-1000 |
| NH₄⁺ (mg/l) | 5-1500 |
| TN (mg/l) | 5-1500 |
| TP (mg/l) | 2-100 |
| ortho-P (mg/l) | 2-100 |

The reduction in the TH obtained after processing by stripping and decarbonation of the vinasses as they are is given as an example in the following table 4.

TABLE 4

| | Stripped and |
|---|---|
| Parameters | decarbonated effluent |
| TH vinasses as they are (° F.) | 0-1000 |
| TH after stripping + decarbonation (° F.) | 0-50 |
| Yield (%) | ≦95% |

A fraction of the stripped and decarbonated effluent (0 to 400% by weight of vinasses and of phlegmas) is recycled to the top of methanization so as to reduce the TH in the reactor, to limit the increases in pH and to be free of the precipitation phenomena which could be unfavorable for the anaerobic biological process.

Furthermore, this stripping-decarbonation step also protects the reverse osmosis filtration step against carbonate mineral precipitation phenomena.

The fraction of the stripped and decarbonated effluent not recycled to the digester is directed to the processing by reverse osmosis in order to reduce, or even virtually completely eliminate, the following parameters: COD, BOD₅, SS, TN, NH₄⁺, NO₃⁻, NO₂⁻, P, Ca²⁺, Mg²⁺, K⁺, Na⁺, and the like, with a view to recycling the processed water.

The concentrations of the effluent after processing by reverse osmosis are given as an example in table 5.

TABLE 5

| | Concentration of the effluent after |
|---|---|
| Parameters | processing by reverse osmosis |
| pH | 5.5-8 |
| TH (° F.) | 0 |
| PA (° F.) | 0-100 |
| TA (° F.) | 0-100 |
| COD (mg/l) | 0-50 |
| SS (mg/l) | 0 |
| NH₄⁺ (mg/l) | 0-200 |
| NO₃⁻ (mg/l) | 0-10 |
| TN (mg/l) | 0-200 |
| TP (mg/l) | 0-10 |
| ortho-P (mg/l) | 0-10 |
| K⁺ (mg/l) | <50 |
| Na⁺ (mg/l) | <50 |
| SO₄²⁻ (mg/l) | <100 |

The processing scheme (sum of all the processing) as defined, namely preprocessing of the fermented wort before extraction, processing by methanization, processing by stripping-decarbonation, and recycling of a fraction of the stripped and decarbonated effluent to the top of methanization, allows an increase in the inhibitory limiting values for the biological processes, in the raw materials, compared with the values normally defined for processing by methanization.

This increase in limiting threshold is possible owing to the high performance level of the elimination processing operations carried out.

Tests carried out by the applicants have shown that the characteristics of vinasses after distillation of fermented worts are strongly influenced by the composition of the fermented worts and, in particular, by the concentration of SS; indeed, the SS of the worts, consisting mainly of organic matter, will, during the distillation, be brought to a high temperature and a significant part will be modified and solubilized (for example, Maillard reactions leading to the formation of nonbiodegradable, or even toxic, compounds); other compounds such as, for example, proteins, amino acids, nitrogen and organic phosphorus contributing to the colloidal and soluble fraction of the fermented worts before distillation will also undergo the effects of temperature (for example, Maillard reactions) leading to the formation of nonbiodegradable, or even toxic, compounds. This results, after distillation in the presence of these SS and colloids, in very different vinasse biodegradability characteristics which very negatively and strongly influence:
firstly, the proportioning of the digesters to be used, requiring large volumes, or even the technology to be used, requiring additional and expensive separators,
and secondly, the pollution elimination yield.

The losses of SS and the decrease in yields result in the end in a considerable limitation of the energy production, but also in the high residual pollutant flows being taken into account for the proportioning of the finishing processing that may result in increased costs that are incompatible with the processing being cost-effective and with the in-process recycling of the process water.

The results of the tests carried out are given in the following table 6.

TABLE 6
Influence of vinasse SS on processing

| | | | | |
|---|---|---|---|---|
| SS concentration | 1% | 0.5% | 0.2% | 0.1% |
| COD | 39 g/l | 35.5 g/l | 30.5 g/l | 30 g/l |
| BOD5 | 12 g/l | 12.00 g/l | 12 g/l | 12 g/l |
| COD/BOD5 | 3.25 | 2.96 | 2.54 | 2.5 |
| Methanization yield | 62.8% | 69.8% | 93.4% | 96.7% |
| Residence time in the | >10 days | >10 days | 3 days | 3 days |
| methanization system | | | | |
| Methane production in | 7.9 | 8.1 | 9.98 | 10.15 |
| liter of CH₄ per liter | | | | |
| of vinasse | | | | |
| % energy produced from | 81 | 90 | 96 | 98 |
| the cake obtained by | | | | |
| the separation upstream | | | | |
| of the distillation | | | | |

15

This table 6 reveals the very surprising results obtained with SS contents in the vinasses (COPRODUCT 1) of 0.2% and 0.1%, resulting in residence times in the methanization system of 3 days only, with a methanization yield greater than 90%.

In this table 6, the definition of the methanization yield is given by the following calculation: COD entering−COD exiting: COD entering.

For the definition of the percentage of energy produced from the cake, the theoretical level of 100% corresponds to all of the dry matter initially contained in the worts. By way of example, it is about 55 to 65 kg per hectoliter of alcohol produced.

1.-16. (canceled)

17. A process of methanization, from a liquid phase which is a coproduct resulting from the extraction of a main product obtained from a vegetable raw material, wherein the content by weight of suspended solids (SS) in said liquid phase is less than 0.25%, and the methanization processing of said liquid phase is carried out by means of a very high yield digester.

18. The process as claimed in claim 17, wherein the content by weight of suspended solids (SS) in said liquid phase is less than 0.2%, in particular less than or equal to 0.1%.

19. The process as claimed in claim 17, wherein the very high yield digester is a bed of fluidized, recirculated or expanded granular sludge in one or more stages or of flocculated sludge, according to an upflow of the liquid phase.

20. The process as claimed in claim 19, wherein the methanization processing is carried out according to a pulsed feed of said liquid phase in a digester of the “Anapulse” type.

21. The process as claimed in claim 20, wherein the methanization processing is carried out by means of a reactor containing a fluidized bed of biomass in a digester of the “Anaflux” type.

22. The process as claimed in claim 21, wherein the methanization processing is carried out by means of a reactor containing a fixed bed of biomass on an immobile support in a digester of the “Anafiz” type.

23. The process as claimed in claim 17, wherein the very high yield digester is a reactor containing a fluidized bed of biomass (cultures attached to a mobile free support).

24. The process as claimed in claim 17, wherein the very high yield digester is a reactor containing a fixed bed of biomass (culture attached to an immobile support).

25. The process as claimed in claim 17, wherein at least one effluent resulting from the methanization is subjected to a stripping and decarbonation step, and in that at least one fraction of the decarbonated effluent obtained is recycled to the top of the methanization.

26. The process as claimed in claim 25, wherein said recycled fraction is included between 0 to 400% by weight of the vinasses and of the phlegmas.

27. The process as claimed in claim 25, wherein the fraction of the decarbonated effluent which is not recycled to the top of the methanization is subjected to a filtration suitable for stopping particles having sizes of about 0.2 μm, in particular bacteria.

28. The process as claimed in claim 27, wherein said filtration of the fraction of the decarbonated effluent which is not recycled is carried out by reverse osmosis.

29. The process as claimed in claim 28, wherein the reverse osmosis filtration also produces a retentate which subsequently undergoes an “evaporation-concentration” step, and in that the condensates recovered after evaporation-concentration can be used in the form of water.

30. The process as claimed in claim 27, wherein 40 to 95% of the effluent after methanization, stripping and decarbonation processing and filtration can be recycled in the form of bacteria-free water.

31. The process as claimed in claim 27, wherein before said filtration, said fraction of the decarbonated effluent is subjected to biological processing of the carbonaceous and/or nitrogenous pollution, and/or to physicochemical processing of the phosphorus-containing pollution.

32. The process as claimed in claim 17, wherein carbon dioxide (CO₂), in particular carbon dioxide produced during the process, is used:
for neutralization of lime in order to produce carbonate of lime,
for partial replacement of at least one acid used for said extraction of the main product.
